(19) United States
(12) Patent Application Publication
(10) Pub. No.: US 2024/0156463 A1
(43) Pub. Date: May 16, 2024

(54) IMPLANTABLE MEDICAL DEVICE ADAPTABLE TO IRREGULAR ANATOMY

(71) Applicant: BOSTON SCIENTIFIC SCIMED, INC., MAPLE GROVE, MN (US)

(72) Inventors: Joshua Mark Inouye, Brooklyn Park, MN (US); Jerry Wang, Edina, MN (US); James M. Anderson, Corcoran, MN (US)

(73) Assignee: BOSTON SCIENTIFIC SCIMED, INC., MAPLE GROVE, MN (US)

(21) Appl. No.: 18/509,090

(22) Filed: Nov. 14, 2023

Related U.S. Application Data
(60) Provisional application No. 63/425,465, filed on Nov. 15, 2022.

(51) Int. Cl. A61B 17/12

(52) U.S. Cl. CPC A61B 17/12177; A61B 17/12031; A61B 17/12122; A61B 17/1214; A61B 2017/1205

(57) ABSTRACT

An implantable medical device such as but not limited to a left atrial appendage closure (LAAC) device includes an expandable frame that is movable between a collapsed configuration for delivery and an expanded configuration for deployment. The expandable frame may include a plurality of articulating members that are biased into the expanded configuration. The expandable frame may include a biasing member. The LAAC device includes a membrane or covering that spans across an end of the expandable frame. In some cases, the LAAC device may be adjustable to better fit a non-circular ostium.

### CROSS REFERENCE TO RELATED APPLICATIONS

This application claims the benefit of priority of U.S. Provisional Application No. 63/425,465 filed Nov. 15, 2022, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device that includes an expandable frame moveable between a collapsed configuration for delivery and an expanded configuration for deployment, the expandable frame adjustable when in the expanded configuration between having a circular overall shape and having an ovoid overall shape. A covering spans at least part of the expandable frame. An adjustment mechanism is disposed within the expandable frame, the adjustment mechanism adapted to controllably adjust the expandable frame between the circular overall shape and the ovoid overall shape.

Additionally or alternatively, the adjustment mechanism may be adapted to controllably adjust the expandable frame after the expandable frame has expanded into its expanded configuration.

Additionally or alternatively, the adjustment mechanism may include a jack-screw mechanism.

Additionally or alternatively, the jack-screw mechanism may include a jack-screw, a scissors mechanism engaged with the jack-screw such that rotation of the jack-screw in a first direction causes the scissors mechanism to extend further radially outwardly and rotation of the jack-screw in an opposing second direction causes the scissors mechanism to retract radially inwardly, a first pad disposed at a first radial extent of the scissors mechanism and a second pad disposed at a second radial extent of the scissors mechanism, the first pad and the second pad adapted to push the expandable frame in radially outward direction when the jack-screw is rotated in the first direction.

Additionally or alternatively, the covering may be adapted to accommodate changes in dimensions of the expandable frame when the expandable frame is adjusted between the circular overall shape and the ovoid overall shape.

Additionally or alternatively, the covering may include a web of large fibers and small fibers spanning between the large fibers.

Additionally or alternatively, the large fibers may include more than fifty percent elastomer and the small fibers may include more than fifty percent PET (polyethylene terephthalate).

Additionally or alternatively, the large fibers may include 70 percent elastomer and 30 percent PET.

Additionally or alternatively, the small fibers may include 70 percent PET and 30 percent elastomer.

Additionally or alternatively, the implantable medical device may include a Left Atrial Appendage Closure (LAAC) device.

Additionally or alternatively, the LAAC device may be adapted to fit into an ovoid ostium of an LAA (left atrial appendage) having a first ratio of a long dimension of the ovoid shape and a small dimension of the ovoid shape, and the LAAC device is adapted to achieve the ovoid overall shape having a second ratio of long dimension to short dimension that is greater than the first ratio.

Another example may be found in a Left Atrial Appendage Closure (LAAC) device that includes an expandable frame moveable between a collapsed configuration for delivery and an expanded configuration for deployment, the expandable frame when expanded into the expanded configuration having a non-circular shape in which the expandable frame has a major axis having a major dimension and a minor axis having a minor dimension, the expandable frame having a first ratio equal to the major dimension divided by the minor dimension. A covering spans at least part of the expandable frame.

Additionally or alternatively, the expandable frame may be adapted to be deployed within a non-circular ostium having a major ostium axis having a major ostium dimension and a minor ostium axis having a minor ostium dimension, the non-circular ostium having a native ratio before implantation of the LAAC device equal to the major ostium dimension divided by the minor ostium dimension and a post-deployment ratio that is greater than the native ratio.

Additionally or alternatively, the post-deployment ratio may be less than the first ratio.

Another example may be found in a method for deploying a Left Atrial Appendage Closure (LAAC) device within a Left Atrial Appendage (LAA), the LAA having a non-circular ostium, the non-circular ostium having a first ratio between a major dimension of the non-circular ostium and a minor dimension of the non-circular ostium. The method includes delivering an LAAC device to a position proximate the non-circular ostium, the LAAC adapted to be able to have an expanded configuration in which the LAAC has a second ratio between a major dimension of the LAAC device and a minor dimension of the LAAC device that is greater than the first ratio. The LAAC device is expanded into the expanded configuration in which the LAAC has the second ratio in order to reshape the non-circular ostium into a new shape that is more ovoid than an original shape of the non-circular ostium.

Additionally or alternatively, the LAAC device may be expandable from a collapsed configuration for delivery and an expanded configuration for deployment, and the expanded configuration may provide the second ratio.

Additionally or alternatively, the method may further include disposing a filler material along one or more sides of the LAAC device after expansion.

Additionally or alternatively, the method may further include an initial step of deploying one or more coils within a distal region of the patient's LAA prior to delivering the LAAC device to the position proximate the non-circular ostium.

Additionally or alternatively, expanding the LAAC device from the collapsed configuration for delivery and the expanded configuration for deployment may further include actuating an actuation mechanism in order for the LAAC device to achieve the second ratio.

Additionally or alternatively, the actuation member may include a jack-screw and a scissors mechanism engaged with the jack-screw such that rotation of the jack-screw in a first direction causes the scissors mechanism to extend further radially outwardly and rotation of the jack-screw in an opposing second direction causes the scissors mechanism to retract radially inwardly, and actuating the actuation member may include rotating the jack-screw.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional view of an LAA (left atrial appendage);

FIG. 2 is a perspective view of an illustrative LAAC (left atrial appendage closure) device, shown without a covering;

FIG. 3 is a perspective view of the illustrative LAAC device of FIG. 2, including a covering;

FIG. 4A is a cross-sectional view of an illustrative LAAC device that includes an adjustment mechanism for changing an overall shape of the LAAC, shown prior to actuation of the adjustment mechanism;

FIG. 4B is a top view of the illustrative LAAC device of FIG. 4A, shown prior to actuation of the adjustment mechanism;

FIG. 5A is a cross-sectional view of the illustrative LAAC device of FIG. 4A, shown after actuation of the adjustment mechanism;

FIG. 5B is a top view of the illustrative LAAC device of FIG. 4A, shown after actuation of the adjustment mechanism;

FIG. 6 is a schematic view of an illustrative non-circular ostium;

FIG. 7 is a schematic view of using an elliptical LAAC device to close a non-circular ostium;

FIGS. 8A and 8B together show using an elliptical LAAC device as well as an additional filler material to close a non-circular ostium; and

FIGS. 9A, 9B and 9C together provide views of an illustrative membrane material that may be used as a covering on the LAAC devices described herein.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

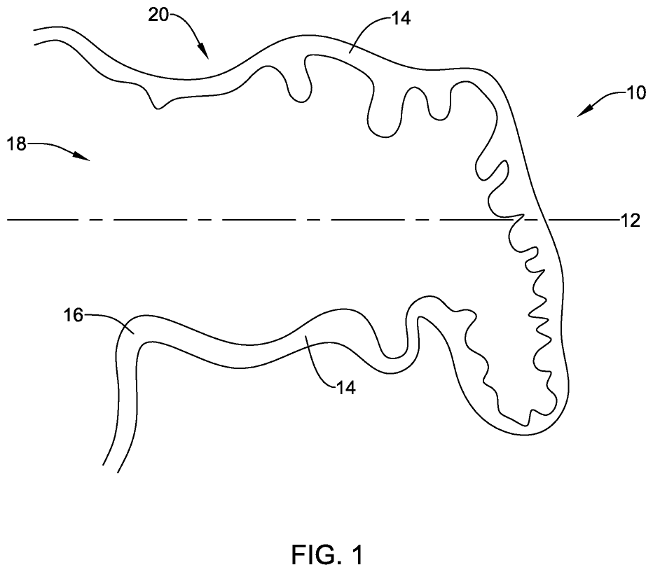

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

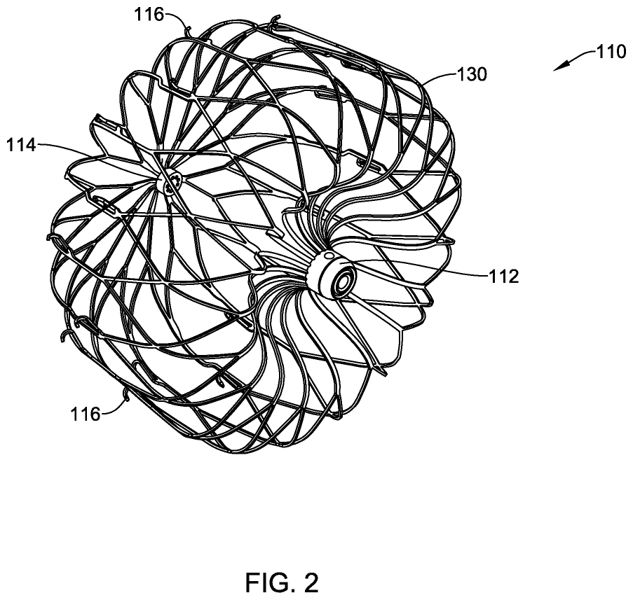

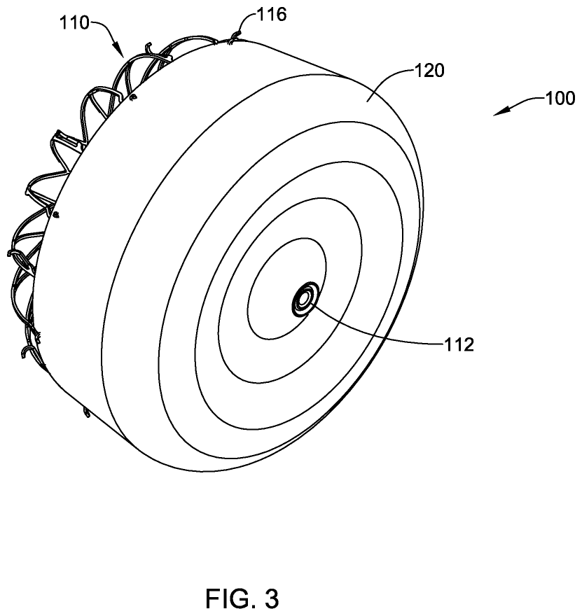

In some instances, a device known as an LAAC (left atrial appendage closure) device may be implanted within the LAA 10, such as near or within the ostium 16, in order to seal off the interior of the LAA 10 from the rest of the heart interior. FIGS. 2 and 3 provide views of a left atrial appendage closure (LAAC) device 100. The LAAC device 100 may include an expandable framework 110 configured to shift axially and/or radially along a central longitudinal axis between the fully constrained configuration and the fully unconstrained configuration. In the fully constrained configuration, the expandable framework 110 may be axially elongated and/or radially compressed. In the fully unconstrained configuration, the expandable framework 110 may be axially shortened and/or radially expanded.

As seen in FIG. 3, which illustrates selected features of the LAAC device 100 in the fully unconstrained configuration, the expandable framework 110 may have a plurality of struts disposed about the central longitudinal axis. In some embodiments, the plurality of struts may define a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements.

The expandable framework 110 may include a proximal hub 112 and a distal hub 114. In some embodiments, the proximal hub 112 and/or the distal hub 114 may be centered on and/or coaxial with the longitudinal axis. The plurality of struts may be joined together at and/or fixedly attached to the proximal hub 112 and/or the distal hub 114. The proximal hub 112 may be configured to releasably connect, secure, and/or attach the left atrial appendage closure device 100 and/or the expandable framework 110 to a delivery device. In some embodiments, the proximal hub 112 may include internal threads configured to rotatably and/or threadably engage an externally threaded distal end of a delivery device. Other configurations for releasably securing the left atrial appendage closure device 100 to a delivery device are also contemplated. As noted herein, some features are not shown in every figure to improve clarity.

The expandable framework 110 and/or the plurality of struts may be formed and/or cut from a tubular member. In some embodiments, the expandable framework 110 and/or the plurality of struts may be integrally formed and/or cut from a unitary member. In some embodiments, the expandable framework 110 and/or the plurality of struts may be integrally formed and/or cut from a unitary tubular member and subsequently formed and/or heat set to a desired shape in the fully unconstrained configuration. In some embodiments, the expandable framework 110 and/or the plurality of struts may be integrally formed and/or cut from a unitary flat member or sheet, and then rolled or formed into a tubular structure and subsequently formed and/or heat set to the desired shape in the fully unconstrained configuration. Some exemplary means and/or methods of making and/or forming the expandable framework 110 and/or the plurality of struts include laser cutting, machining, punching, stamping, electro discharge machining (EDM), chemical dissolution, etc. Other means and/or methods are also contemplated.

As would be understood by the skilled person, anatomical features may vary in size and/or shape. In some embodiments, the left atrial appendage may have an irregular (e.g., elongated and/or oblong) cross-sectional shape. In some embodiments, the expandable framework 110 may be compliant and substantially conform to and/or be in sealing engagement with the shape and/or geometry of a lateral wall of a left atrial appendage when deployed and/or expanded therein. In some embodiments, the left atrial appendage closure device 100 may expand to a size, extent, or shape less than or different from the fully unconstrained configuration, as determined by the surrounding tissue and/or lateral wall of the left atrial appendage. In some embodiments, the expandable framework 110 may be configured to shape and/or stretch the tissue of the left atrial appendage such that the lateral wall of the left atrial appendage substantially conforms to an outer shape of the expandable framework 110. Other configurations are also contemplated.

In some embodiments, the expandable framework 110 may include at least one anchoring member 116 extending radially outward therefrom in the fully unconstrained configuration. In some embodiments, the expandable framework 110 may include at least one anchoring member 116 extending radially outward from the expandable framework 110. In some embodiments, the expandable framework 110 may include at least one anchoring member 116 extending radially outward from the expandable framework 110 near a proximal shoulder of the expandable framework 110. In some embodiments, the expandable framework 110 may include at least one anchoring member 116 extending radially outward from the expandable framework 110 proximate a midsection of the expandable framework 110. In some embodiments, the at least one anchoring member 116 may be configured to engage with the lateral wall of the main body of the left atrial appendage. In some embodiments, the at least one anchoring member 116 may be formed as J-shaped hooks having a free end extending in and/or directed toward a proximal direction with respect to the central longitudinal axis of the left atrial appendage closure device 100 and/or the expandable framework 110. Other configurations are also contemplated.

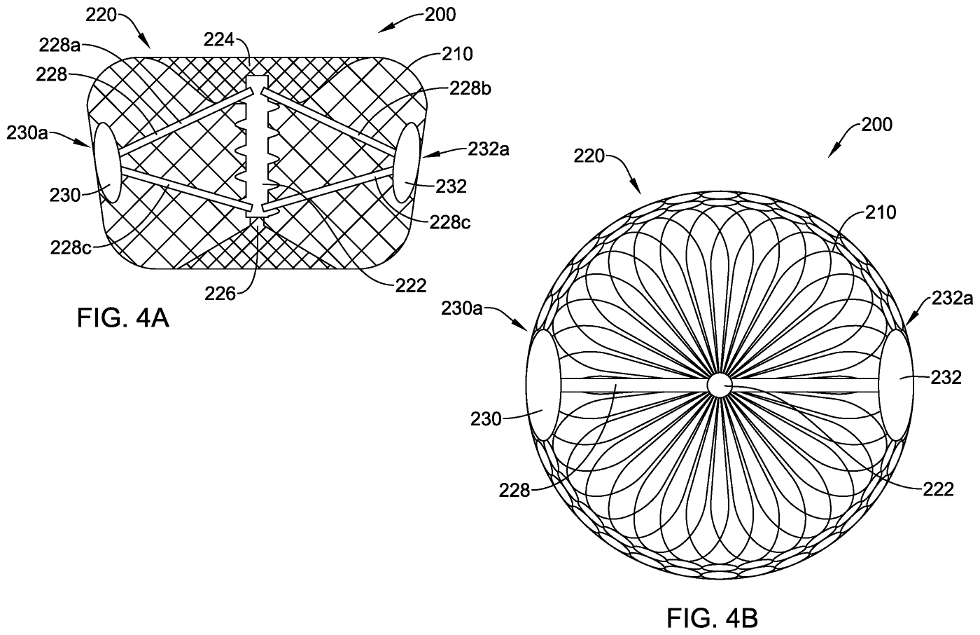

In some embodiments, the left atrial appendage closure device 100 may optionally include the occlusive element 120 connected to, disposed on, disposed over, disposed about, and/or disposed radially outward of at least a portion of the expandable framework 110 and/or the plurality of struts, as seen in FIG. 4. In some embodiments, the occlusive element 120 may be attached to the proximal hub 112 and/or may be attached to the expandable framework at the proximal hub 112. In some embodiments, the occlusive element 120 may extend radially outward from and/or may extend distally from the proximal hub 112. In some embodiments, the occlusive element 120 may be attached and/or secured to the expandable framework 110 at a plurality of discrete locations. In some embodiments, one of, some of, and/or all of the at least one anchoring member 116 may extend through an occlusive element 120, where present.

In some embodiments, the occlusive element 120 may include a membrane, a fabric, a mesh, a tissue element, or another suitable construction. In some embodiments, the occlusive element 120 may be porous. In some embodiments, the occlusive element 120 may be non-porous. In some embodiments, the occlusive element 120 may be permeable to selected gases and/or fluids. In some embodiments, the occlusive element 120 may be substantially impermeable to selected gases and/or fluids, such as blood, water, etc. In some embodiments, the occlusive element 120 may be designed, sized, and/or configured to prevent thrombus and/or embolic material from passing out of the left atrial appendage into the left atrium and/or the patient's bloodstream. In some embodiments, the occlusive element 120 may be configured to promote endothelization after implantation, thereby effectively removing the target site (e.g., the left atrial appendage, etc.) from the patient's circulatory system. Some suitable, but non-limiting, examples of materials for the occlusive element 120 are discussed below.

FIG. 4A is a cross-sectional view of an illustrative LAAC device 200. The LAAC device 200 may be considered as being similar to the LAAC device 100, apart from including an adjustment mechanism 220. The LAAC device 200 may include an expandable framework 210. The expandable framework 210 may be expandable between a collapsed configuration for delivery (not shown) and an expanded configuration for deployment (as shown in FIG. 4A). While not shown, it will be appreciated that the LAAC device 200 may include a covering such as the occlusive element 120 shown and discussed with respect to FIG. 3, for example.

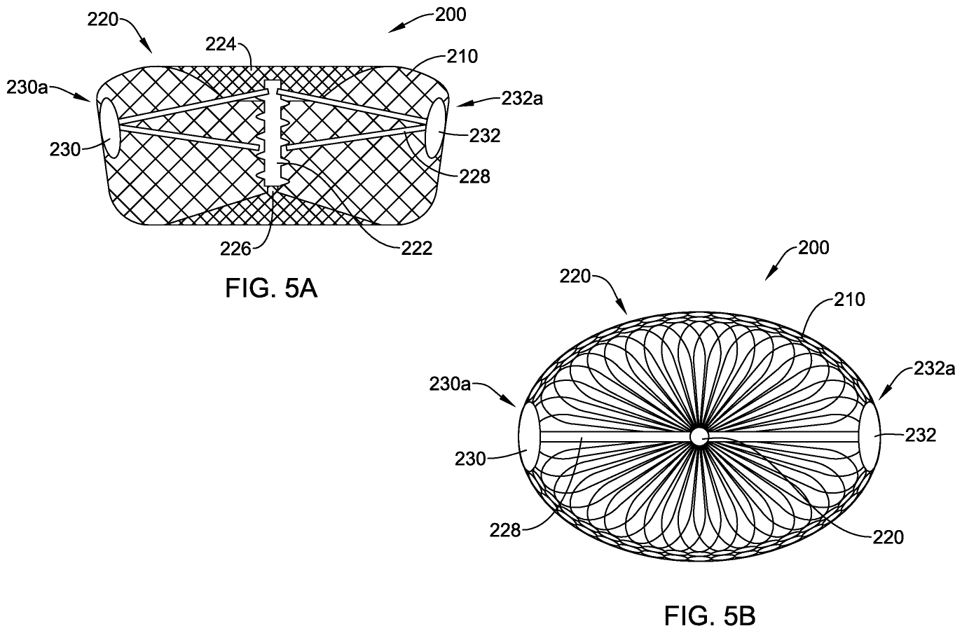

The adjustment mechanism 220 may be adapted to alter an overall shape of the LAAC device 200 from a generally round or circular profile, as shown in FIGS. 4A and 4B, to a generally non-circular or ovoid profile, as shown in FIGS. 5A and 5B. The adjustment mechanism 220 may be considered as being a jack-screw mechanism. In some cases, the adjustment mechanism 220 includes a jack-screw 222 that extends from a proximal hub 224 to a distal hub 226. In some cases, the jack-screw 222 may be adapted to allow rotation of the jack-screw 222 by coupling an elongate tool similar to a screwdriver or socket to an end of the jack-screw 222 proximate the proximal hub 224.

The adjustment mechanism 220 includes a scissors mechanism 228 that is engaged with the jack-screw 222 in a manner that permits rotation of the jack-screw 222 to cause the scissors mechanism 228 to move radially. Rotating the jack-screw 222 in a first direction may cause the scissors mechanism 228 to lengthen, as shown in FIGS. 5A and 5B. Rotating the jack-screw 222 in an opposing, second, direction may cause the scissors mechanism 228 to shorten. The scissors mechanism 228 may include a first pair of scissor members 228a and 228b that are coupled to the jack-screw 222 in a manner that holds the first pair of scissor members 228 and 228b stationary with respect to the jack-screw 222, apart from allowing the jack-screw 222 to rotate relative to the first pair of scissor members 228a and 228b. The scissors mechanism 228 may include a second pair of scissor members 228c and 228d that are coupled to the jack-screw 222 in a manner that allows the jack-screw 222 to engage the second pair of scissor members 228c and 228d in a threaded fashion that means that rotation of the jack-screw 222 causes the second pair of scissor members 228 and 228d to move up or down relative to the jack-screw 222, thereby causing the scissors mechanism 228 to either shorten or lengthen.

In some cases, the LAAC device 200 includes a first pad 230 that is disposed at a first radial extent 230a of the scissors mechanism 228 and a second pad 232 that is disposed at a second radial extent 232a of the scissors mechanism 228. The first pad 230 and the second pad 232 are each adapted to push the expandable framework 210 in a radially outward direction when the jack-screw 222 is rotated in the first direction. The first pad 230 and the second pad 232 may be formed of any suitable material, including an elastomeric polymer, or an elastomeric polymer composite that includes stiffer elements. The stiffer elements may include radiopaque fillers and elements, for example. In some cases, the elastomeric properties of the first pad 230 and the second pad 232 allow the first pad 230 and the second pad 232 to form a grip into the expandable framework 210.

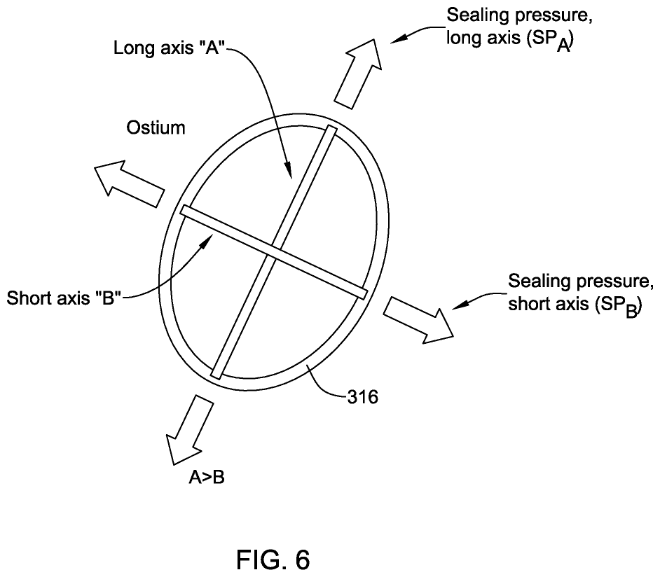

FIGS. 4A-4B and 5A-5B show an example of an LAAC device 200 that is adapted to allow a physician or other professional installing the LAAC device 200 to actively change its shape in order to better fit an ostium 16 that is not circular, but is more of an oval shape. In some cases, an LAAC device may not permit actively changing its shape to better fit the ostium 16, but instead may be adapted to passively change the shape of the ostium 16 without undergoing any changes in dimensions itself. FIG. 6 shows an example ostium 316 that is not circular in shape, but is more ovoid or even elliptical in shape. The ostium 316 has a long axis “A” and a short axis “B” that is orthogonal to the long axis “A”. An elliptical LAAC device (not shown) placed within the ostium 16 may be considered as providing a sealing pressure SPA along the long axis “A” and a sealing pressure SPB along the short axis “B”. In some cases, a good sealing pressure SP A is beneficial to help seal along the mitral area, which is found along the lower left (in the illustrated orientation) of the ostium 316.

In some cases, a ratio may be used to describe how non-circular the ostium 316 and how non-circular (or how elliptical) a corresponding LAAC device may be. For example, the ratio may be referred to simply as A/B, or dividing a dimension along the long axis “A” by a dimension along the short axis “B”. In some cases, an LAAC device may have a larger A/B ratio than that of the native ostium 316. In some cases, the LAAC device, when deployed, may actually increase the A/B ratio of the native ostium. As a result, the ostium 316 may have a final A/B ratio that is intermediate its native A/B ratio and the A/B ratio of the LAAC device that is implanted within the ostium 316.

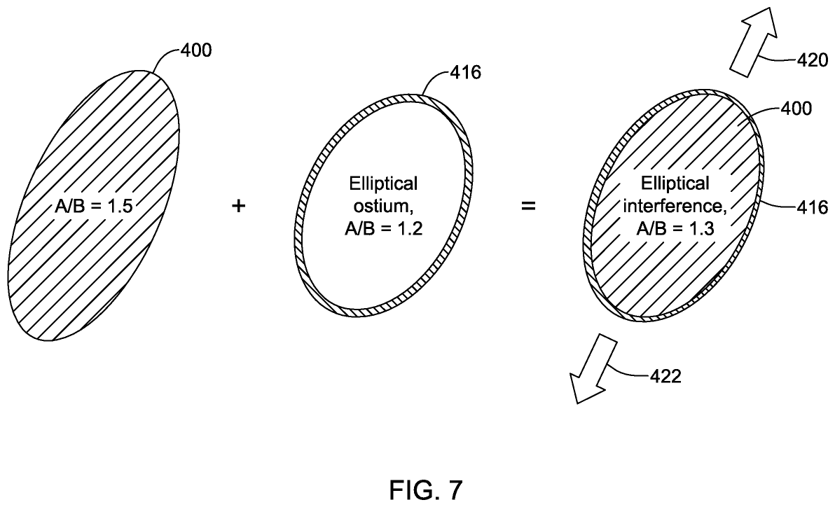

FIG. 7 provides an example of this. An elliptical LAAC device 400 has an A/B ratio of 1.5, meaning that the length of its long axis “A” is 1.5 times the length of its short axis “B”. The elliptical LAAC device 400 is inserted into an ostium 416, similar to the ostium 316, that has an A/B ratio of 1.2, meaning that the length of the long axis “A” of the ostium 416 is 1.2 times that of the length of the short axis “B” of the ostium 416. The LAAC device 400 reshapes the ostium 416 such that the reshaped ostium 416 now has an A/B ratio of 1.3. It will be appreciated that the A/B ratio of the reshaped ostium 416 of 1.3 is intermediate the A/B ratio of the native ostium and the A/B ratio of the LAAC device 400. As a result of reshaping the ostium 416 in this manner, an increased sealing pressure is applied along the long axis “A”, as indicated by arrows 420 and 422. Reshaping the ostium 416 in this manner can result in a high sealing pressure SP A along the long axis “A” and possibly a lower sealing pressure SPB along the short axis “B”. However, the sealing pressure along the long axis “A” has been found to be more important in reducing or eliminating leaks around the LAAC device 400. In some instances, it has been found that leaks, when they occur, are primarily along the long axis ‘A” of the ostium 416. In some cases, the mitral side of the long axis “A” may cause the long axis “A” to lengthen during the cardiac cycle, leading to more ostial motion along the long axis “A”. It is believed that increasing the chronic pressure on the long axis “A” will help to preserve sealing during the long axis motion that occurs during the cardiac cycle.

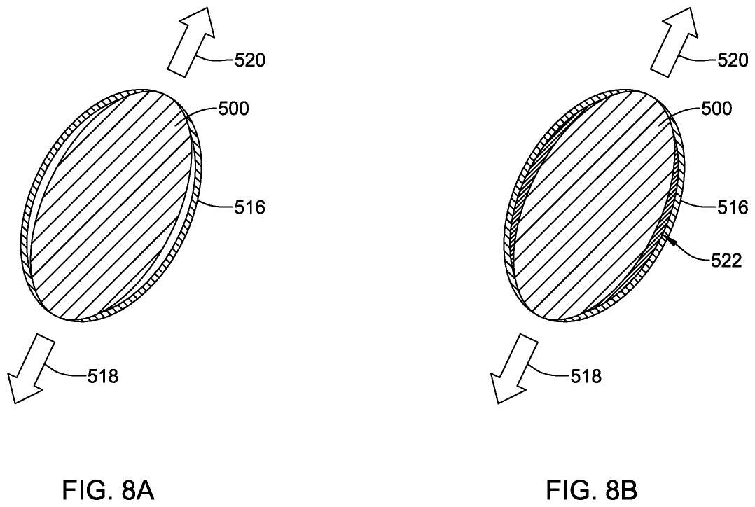

In some cases, the LAAC device itself may not be sufficient. For example, in some cases the elliptical LAAC device may not seal all the way around the ostium. FIG. 8A shows an LAAC device 500 that has been disposed within an ostium 516. While the LAAC device 500 has at least partially reshaped the ostium 516, and looks to be applying a good sealing pressure SP A along the long axis “A”, as indicated by arrows 518 and 520, there are gaps between the LAAC device 500 and the ostium 516 along the short axis “B”. In some cases, a filler material 522 may be provided to fill in the gaps along the sides between the LAAC device 500 and the ostium 516 to help seal against leaks. The filler material 522 may be a foam, for example, or a hydrogel. The filler material 522 may be a coil or a fabric filler. Adding the filler material 522 allows for occlusion even though the sealing pressure along the short axis “B” is perhaps lower than would ideally be wanted.

It will be appreciated that in some cases, an LAAC device may be deployed within an LAA 10 in order to help seal off the LAA 10 from the rest of the interior of the heart, even if the ostium 16 is non-circular. The ostium may be considered as having an A/B ratio as previously discussed. An LAAC device may be delivered to a position proximate the non-circular ostium. The LAAC device may be considered as being adapted to be able to have an expanded configuration in which the LAAC has a second ratio between a major dimension of the LAAC device and a minor dimension of the LAAC device that is greater than the first ratio.

The LAAC device is expanded into the expanded configuration in which the LAAC has the second ratio in order to reshape the non-circular ostium into a new shape that is more ovoid than an original shape of the non-circular ostium. In some cases, the LAAC device is expandable from a collapsed configuration for delivery and an expanded configuration for deployment, and the expanded configuration provides the second ratio.

In some cases, the method may further include disposing a filler material along one or more sides of the LAAC device after expansion. In some cases, the method may further include an initial step of deploying one or more coils within a distal region of the LAA prior to delivering the LAAC device to the position proximate the non-circular ostium. In some cases, adding one or more coils within the distal region of the LAA can help prevent tilting of the LAAC device, for example.

In some cases, expanding the LAAC device from the collapsed configuration for delivery and the expanded configuration for deployment may further include actuating an actuation mechanism in order for the LAAC device to achieve the second ratio. As an example, the actuation member may include a jack-screw and a scissors mechanism engaged with the jack-screw such that rotation of the jack-screw in a first direction causes the scissors mechanism to extend further radially outwardly and rotation of the jack-screw in an opposing second direction causes the scissors mechanism to retract radially inwardly, wherein actuating the actuation member includes rotating the jack-screw.

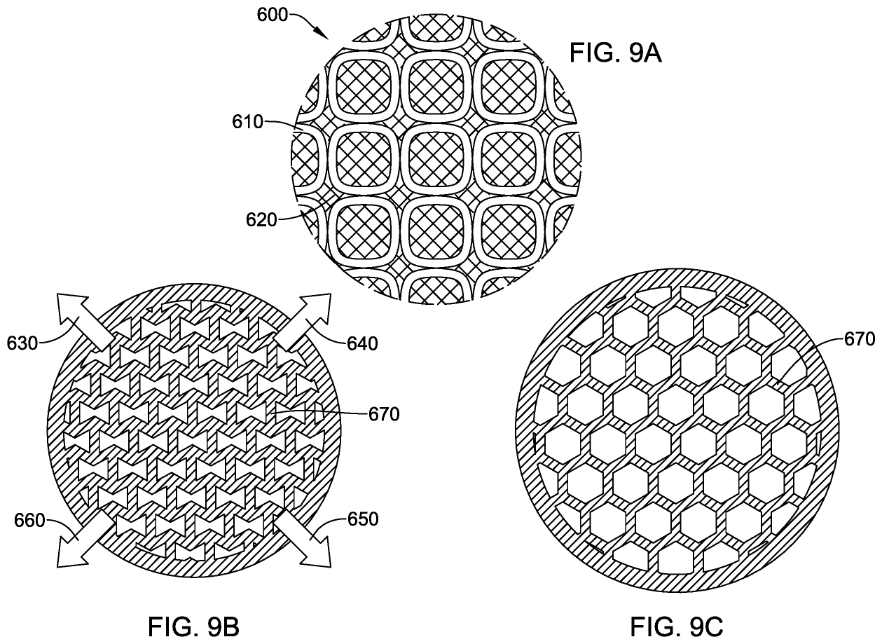

As will be appreciated, in some cases the covering (such as the occlusive element 120) that spans the expandable frame 110 may have to accommodate changes in the dimensions of the expandable frame 110. In other words, the covering may have to be able to stretch. FIGS. 9A, 9B and 9C together provide details of a covering 600 that may be used with the LAAC devices described herein. The covering 600 includes a webbing 610 that is made from a relatively thick fiber. A webbing 620 spans the distance between the relatively thick fibers forming the webbing 610 and is formed from relatively thin fibers. In some cases, the webbing 610 may be formed of fibers having an average diameter in a range of 5 to 10 μm and the webbing 620 may be formed of fibers having an average diameter in a range of 25 to 100 μm. In some cases, the webbing 610 is laid out in a honeycomb fashion, but this is not required in all cases.

In some cases, the webbing 610 may be formed of fibers that have a relatively large fraction of elastomer and a relatively smaller fraction of a second polymer such as but not limited to PET (polyethylene terephthalate). In some cases, the webbing 610 may be formed of fibers that are at least 50 percent elastomer and the webbing 620 may be formed of fibers that are at least 50 percent PET. In some cases, the webbing 610 may be formed of fibers that include about 70 percent elastomer and about 30 percent PET. In some cases, the webbing 620 may be formed of fibers that have a relatively large fraction of PET and a relatively smaller fraction of an elastomer. In some cases, the webbing 620 may be formed of fibers that include about 30 percent PET and about 70 percent elastomer. The elastomers used in the webbing 610 and the webbing 620 may include one or more of fluoroelastomers, polyurethane elastomers, Pebax, thermoplastic elastomers, copolyester elastomer, hydrophilic elastomers, polyamide 11 or polyether segments.

FIGS. 9B and 9C together illustrate how the covering 600 responds to applied forces. In particular, FIGS. 9B and 9C together show that tension applied in any direction, as indicated by the arrows 630, 640, 650 and 660, result in equal porosity. In FIG. 9C, the mesh 670 can be seen as having equal pore sizes. In some cases, the covering 600 may be considered as exhibiting auxetic properties. In some cases, the covering 600 may include materials such as urethane or nylon. In some cases, the covering 600 may also include radiopaque elements. In some cases, the covering 600 may be a fabric matrix that is formed in an auxetic pattern, such that stretching and compliance in a planar radial axis is equalized and distributed consistently regarding porosity of hemodynamic flow as well as hemostasis.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
an expandable frame moveable between a collapsed configuration for delivery and an expanded configuration for deployment, the expandable frame adjustable when in the expanded configuration between having a circular overall shape and having an ovoid overall shape;
a covering spanning at least part of the expandable frame; and
an adjustment mechanism disposed within the expandable frame, the adjustment mechanism adapted to controllably adjust the expandable frame between the circular overall shape and the ovoid overall shape.

2. The implantable medical device of claim 1, wherein the adjustment mechanism is adapted to controllably adjust the expandable frame after the expandable frame has expanded into its expanded configuration.

3. The implantable medical device of claim 1, wherein the adjustment mechanism comprises a jack-screw mechanism.

4. The implantable medical device of claim 3, wherein the jack-screw mechanism comprises:
a jack-screw;
a scissors mechanism engaged with the jack-screw such that rotation of the jack-screw in a first direction causes the scissors mechanism to extend further radially outwardly and rotation of the jack-screw in an opposing second direction causes the scissors mechanism to retract radially inwardly; and
a first pad disposed at a first radial extent of the scissors mechanism and a second pad disposed at a second radial extent of the scissors mechanism, the first pad and the second pad adapted to push the expandable frame in radially outward direction when the jack-screw is rotated in the first direction.

5. The implantable medical device of claim 1, wherein the covering is adapted to accommodate changes in dimensions of the expandable frame when the expandable frame is adjusted between the circular overall shape and the ovoid overall shape.

6. The implantable medical device of claim 5, wherein the covering comprises:
a web of large fibers; and
small fibers spanning between the large fibers.

7. The implantable medical device of claim 6, wherein the large fibers comprise more than fifty percent elastomer and the small fibers comprise more than fifty percent PET (polyethylene terephthalate).

8. The implantable medical device of claim 6, wherein the large fibers comprise 70 percent elastomer and 30 percent PET.

9. The implantable medical device of claim 6, wherein the small fibers comprise 70 percent PET and 30 percent elastomer.

10. The implantable medical device of claim 1, wherein the implantable medical device comprises a Left Atrial Appendage Closure (LAAC) device.

11. The implantable medical device of claim 10, wherein the LAAC device is adapted to fit into an ovoid ostium of an LAA (left atrial appendage) having a first ratio of long dimension of the ovoid shape and a small dimension of the ovoid shape, and the LAAC device is adapted to achieve the ovoid overall shape having a second ratio of long dimension to short dimension that is greater than the first ratio.

12. A Left Atrial Appendage Closure (LAAC) device, comprising:
an expandable frame moveable between a collapsed configuration for delivery and an expanded configuration for deployment, the expandable frame when expanded into the expanded configuration having a non-circular shape in which the expandable frame has a major axis having a major dimension and a minor axis having a minor dimension, the expandable frame having a first ratio equal to the major dimension divided by the minor dimension; and
a covering spanning at least part of the expandable frame.

13. The LAAC device of claim 12, wherein the expandable frame is adapted to be deployed within a non-circular ostium having a major ostium axis having a major ostium dimension and a minor ostium axis having a minor ostium dimension, the non-circular ostium having a native ratio before implantation of the LAAC device equal to the major ostium dimension divided by the minor ostium dimension and a post-deployment ratio that is greater than the native ratio.

14. The LAAC device of claim 13, wherein the post-deployment ratio is less than the first ratio.

15. A method for deploying a Left Atrial Appendage Closure (LAAC) device within a Left Atrial Appendage (LAA), the LAA having a non-circular ostium, the non-circular ostium having a first ratio between a major dimension of the non-circular ostium and a minor dimension of the non-circular ostium, the method comprising:
delivering an LAAC device to a position proximate the non-circular ostium, the LAAC adapted to be able to have an expanded configuration in which the LAAC has a second ratio between a major dimension of the LAAC device and a minor dimension of the LAAC device that is greater than the first ratio; and
expanding the LAAC device into the expanded configuration in which the LAAC has the second ratio in order to reshape the non-circular ostium into a new shape that is more ovoid than an original shape of the non-circular ostium.

16. The method of claim 15, wherein the LAAC device is expandable from a collapsed configuration for delivery and an expanded configuration for deployment, and the expanded configuration provides the second ratio.

17. The method of claim 16, further comprising disposing a filler material along one or more sides of the LAAC device after expansion.

18. The method of claim 15, further comprising an initial step of deploying one or more coils within a distal region of the LAA prior to delivering the LAAC device to the position proximate the non-circular ostium.

19. The method of claim 15, wherein expanding the LAAC device from the collapsed configuration for delivery and the expanded configuration for deployment further comprises actuating an actuation mechanism in order for the LAAC device to achieve the second ratio.

20. The method of claim 19, wherein the actuation member comprises:
a jack-screw and a scissors mechanism engaged with the jack-screw such that rotation of the jack-screw in a first direction causes the scissors mechanism to extend further radially outwardly and rotation of the jack-screw in an opposing second direction causes the scissors mechanism to retract radially inwardly;
wherein actuating the actuation member comprises rotating the jack-screw.
